(19) United States
(12) Patent Application Publication
(10) Pub. No.: US 2021/0186468 A1
(43) Pub. Date: Jun. 24, 2021

(54) VARIABLE DENSITY SPATIAL SCANNING AND ELECTRO-MECHANICALLY CONTROLLED ULTRASOUND SCAN DENSITY DEVICE

(71) Applicant: Accutome, Inc., Malvern, PA (US)

(72) Inventors: Mark E. Schafer, Lower Gywnedd, PA (US); Cheng-Ning Chang, North Wales, PA (US); James Wesley Barnham, London (GB); Marek Gumienny, Slough (GB); Ross Lefkowitz, Philadelphia, PA (US)

(73) Assignee: Accutome, Inc., Malvern, PA (US)

(21) Appl. No.: 17/131,163

(22) Filed: Dec. 22, 2020

Related U.S. Application Data
(60) Provisional application No. 62/952,842, filed on Dec. 23, 2019.

(51) Int. Cl. A61B 8/00

(52) U.S. Cl. CPC A61B 8/54; A61B 8/4461

(57) ABSTRACT

An ultrasound system includes a computing device, a transducer configured to angulate through a scan region in response to a mechanical drive system that converts rotational motion generated by a motor into angular motion that angulates the transducer through the scan region, and an encoder configured to detect a rotational position of a shaft of the motor. The computing device is configured to determine an angular position of the transducer within the scan region based on the rotational position of the shaft detected by the encoder, and control generation of scan lines from the transducer based on a pulse firing pattern of scan lines to produce a predefined sequence of scan line densities across the scan region and the determined angular position of the transducer.

### CROSS-REFERENCE TO RELATED APPLICATION

This application claims the benefit of U.S. Provisional Application No. 62/952,842, filed Dec. 23, 2019, the entirety of which is hereby incorporated by reference.

### TECHNICAL FIELD

The present specification generally relates to ultrasonics, specifically ultrasound medical imaging. More specifically, the subject matter of the present disclosure relates to ultrasound medical imaging of the eye and nearby structures.

### BACKGROUND

Ultrasound medical scanning has become a ubiquitous procedure and has provided a significant benefit to patients. In the field of ophthalmology, the ability to determine internal eye structures when the normal optical path is blocked by, for example, a cataract, provides physicians with increased assurances when making diagnoses and performing treatments such as surgical operations. Ultrasound scanning can be used for surgical treatment planning in the case of undisclosed conditions which would impact cataract surgery. Additionally, ophthalmic ultrasound scanning may be used to image ocular tumors, detect foreign bodies in the eye, and/or quantify detached retinas.

### SUMMARY

In one embodiment, an ultrasound system includes a computing device, a transducer configured to angulate through a scan region in response to a mechanical drive system that converts rotational motion generated by a motor into angular motion that angulates the transducer through the scan region, and an encoder configured to detect a rotational position of a shaft of the motor. The computing device is configured to determine an angular position of the transducer within the scan region based on the rotational position of the shaft detected by the encoder, and control generation of scan lines from the transducer based on a pulse firing pattern of scan lines to produce a predefined sequence of scan line densities across the scan region and the determined angular position of the transducer.

In some embodiments, a method of controlling an ultrasound device including a transducer configured to angulate through a scan region in response to a mechanical drive system that converts rotational motion generated by a motor into angular motion that angulates the transducer through the scan region, and an encoder configured to detect a rotational position of a shaft of the motor is disclosed. The method includes determining an angular position of the transducer within the scan region based on the rotational position of the shaft detected by the encoder; and controlling generation of scan lines from the transducer based on a pulse firing pattern of scan lines to produce a predefined sequence of scan line densities across the scan region and the determined angular position of the transducer.

In some embodiments, an ultrasound device includes a motor having a shaft configured to generate rotational motion, an encoder configured to detect a rotational position of the shaft of the motor, a cam rotatably coupled to the shaft of the motor, the cam comprising a cam profile defining a contour about a socket, the socket configured to receive a ball portion of a cam follower, one or more cam follower contacts positioned on the cam follower to contact the cam profile of the cam, and a transducer coupled to the cam follower, the transducer configured to angulate through a scan region in response the rotational motion generated by the motor that causes the cam to rotate and the cam follower to angulate back and forth about a pivot in response to an interface between the one or more cam follower contacts and the cam profile of the cam.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and in the claims, the singular form of ‘a’, ‘an’, and ‘the’ include plural referents unless the context clearly dictates otherwise.

### BRIEF DESCRIPTION OF THE DRAWINGS

The embodiments set forth in the drawings are illustrative and exemplary in nature and are not intended to limit the subject matter defined in the claims. The following detailed description of the illustrative embodiments can be understood when read in conjunction with the following drawings, where like structures are indicated with like reference numerals and in which:

FIG. 1 depicts an illustrative example of an ophthalmic ultrasound image according to one or more embodiments shown and described herein;

FIG. 2 depicts an illustrative motion pattern of an ultrasound sector scan probe according to one or more embodiments shown and described herein;

FIG. 3A depicts a graphical representation of different scan line density plots as a function of angular position of the transducer within the scan region according to one or more embodiments shown and described herein;

FIG. 3B depicts another graphical representation of different scan line density plots as a function of angular position of the transducer within the scan region according to one or more embodiments shown and described herein;

FIG. 4 schematically depicts an ultrasound device implementing a first mechanical drive system which angulates the transducer about a pivot using a motor which creates a rotary motion according to one or more embodiments shown and described herein;

FIG. 5 schematically depicts another ultrasound device implementing a second mechanical drive system which angulates the transducer about a pivot according to one or more embodiments shown and described herein;

FIG. 6 schematically depicts another ultrasound device implementing a third mechanical drive system which angulates the transducer about a pivot according to one or more embodiments shown and described herein;

FIGS. 7A-7H depict schematic representations of a portion of an ultrasound device implementing a mechanical drive system having a cam and a cam follower configured to receive rotational motion from a motor and convert the rotational motion into angular motion that angulates the transducer about a pivot according to one or more embodiments shown and described herein;

FIGS. 8A-8C depict cross-sectional representations of the ultrasound device shown in FIG. 7A according to one or more embodiments shown and described herein;

FIG. 9 depicts an illustrative schematic of a partial assembled view of the ultrasound device according to one or more embodiments shown and described herein;

FIG. 10 depicts a block diagram of a means of using encoder signals to produce a transducer pulse firing pattern that is corresponds to the angular position of the transducer according to one or more embodiments shown and described herein;

FIG. 11 depicts an illustrative ultrasound system, according to one or more embodiments shown and described herein;

FIG. 12 depicts an illustrative method of implementing an ultrasound scan according to one or more embodiments shown and described herein;

FIG. 13A depicts a plot of pulse firing events using the typical mechanical system; and

FIG. 13B depicts a plot depicting the electro-mechanically controlled ultrasound scan density device according to one or more embodiments shown and described herein.

### DETAILED DESCRIPTION

Embodiments of the present disclosure are directed to systems, methods, and devices configured to detect the angular position of an ultrasound transducer and adjust the pulse firing pattern of the scan lines to produce a predefined sequence of scan line densities across a scan region. Embodiments also include means for resolving the small structures within the eye, which often requires that the ultrasound system operate at a relatively high frequency, such as 12-20 MHz, by controlling the pulse firing patterns of the scan lines to generate uniform or predefined scan line densities across a scan region. The overall system design is made to be as simple as possible to meet cost constraints so that the system and device may serve the largest number of patients. The present disclosure contemplates that mechanically scanned sector probes have the capacity to provide workable solutions to these challenges by implementing streamlined mechanical drive mechanisms and methods of controlling the same.

Mechanically scanned sector ultrasound probes contemplated by the present disclosure may have a single element transducer, a multiple element annular array transducer, or a multiple element linear array, any of which may be mechanically steered or pivoted over an angular range in a pattern called a “sector scan”. As the transducer is swept, it transmits a short pulse of ultrasound energy. Each firing or pulsing of the ultrasound transducer produces echo data along the line of the ultrasound beam. These are called “scan lines,” as they are lines of echo data that make up the overall ultrasound image, or scan.

The spatial resolution of the image is controlled by several factors. The resolution along the scan line, referred to as “axial” resolution, is set by the temporal resolution of the ultrasound pulse from the transducer. The resolution in the direction perpendicular to the axis of a scan line, which is referred to as “lateral” resolution, is set by the beam characteristics of the transducer. A further factor affecting image quality is the density of the scan lines within in the sector scan. That is, the more scan lines within a fixed scan angle, the “denser” the data available for image reconstruction, and the better the image quality. However, there are practical limits to the number of scan lines because of the extra demand on the hardware for data transfer, and especially on the software algorithms implemented for image reconstruction. Image reconstruction algorithms are used to collect and convert the data from individual scan lines into a diagnostic image. Therefore, it is not practical to simply increase the number of scan lines per image to improve image quality.

In a classic mechanically scanned ultrasound system, the scan line density may not be uniform with respect to angular position. As used herein, the term “uniform” refers to uniformity within a predefined tolerance. As the transducer is angulated, the ultrasound transducer is fired and scan lines are created at regular temporal intervals, referred to as the Pulse Repetition Interval (“PRI”). The inverse of the PRI is called the Pulse Repetition Frequency (“PRF”). Maintaining a constant or near constant pulsing pattern simplifies the system design because all the timing issues involved with data acquisition and transfer are constant. As used herein, the term “constant” refers to maintaining a value at a predefined value and/or within a predefined range.

However, the mechanical angular position of the transducer may not be uniform in time over the entire scan region. Mechanical ultrasound scanners have been configured such that the transducer rotates in a single direction at a constant speed. This insures that the change in angular position between scan lines is the same throughout the image, assuming that the transducer is pulsed at a constant temporal rate. However, this requires that the transducer be connected electrically using brushes or inductive means. This type of system is considered obsolete because of the low sensitivity that results from inductive coupling, or the high noise and reliability issues caused by using brushes.

Other mechanical ultrasound scanners may implement a transducer that is angled back and forth. As such, the transducer can be directly connected via a flexible wire. However, in this type of scanner the transducer must naturally slow down at the edges of the scan region because it needs to stop and reverse direction. Thus, if uniform temporal pulsing is employed, there will be more scan lines at the edge of the image than at the center, because the angular speed of the transducer is slower at the edges of the image. Further, it is common that the angular speed of the transducer is never constant, and is always slightly accelerating or decelerating.

To address this issue, attempts can be made to compensate for the variation in angular speed by varying pulsing times. For instance, pulsing may occur at regular angular intervals rather than at regular time intervals. This produces a uniform angular distribution of the scan data. This may be an appropriate approach for many types of ultrasound scanning, especially since, in general, the area of interest in the ultrasound scan is not known beforehand and all regions of the entire scan area should be considered equally important. This approach is also simpler in terms of image reconstruction, which may include converting data from a scan line representation to a video or image representation, since the angular interval between each scan line is the same throughout the image. However, this requires very precise timing control as well as very precise knowledge of the angular position of the transducer. This additional precision can drive up the complexity and cost of the system.

Ophthalmic ultrasound imaging presents a different use case than most other ultrasound applications. That is, the image configuration is generally the same for the majority of scans. The ultrasound probe is placed on the front of the eye, for example, on the cornea, and the mechanical sector region encompasses the orbit and the rear of the eye. The area to be imaged, which appears generally as a circular structure, is uniform from patient to patient. It is also symmetric about the central axis, that is, about a line drawn from the center of the probe to the center of the back of the eye. In addition, the image is generally consistent in that the central angular region of the image has the cornea, lens, vitreous, and the retina in the back of the eye. The sclera, which appears at the edges of the image, may be of lesser importance in general.

Thus if a mechanical sector scanner with an angulating transducer uses a uniform temporal pulse rate, it would have a higher scan line density and therefore higher image quality at the edges of the scan where the angulation rate was the slowest, which corresponds to the region of the eye that may be of the least interest, such as the sclera. More importantly, if the scanning mechanism produced non uniform angular velocity throughout the scan, which is common, then the angular interval between scan lines would not be uniform. Therefore, there is need for a scanner, system, and method that provides uniform scan line density in the central region of the image compared to the edges. This would preserve diagnostic information in regions which are of higher medical interest while keeping cost and complexity low.

It would therefore be beneficial to have a method or means to provide uniform scan line density in the central region of an ultrasound scan taken with an angulating mechanical sector ultrasound scanner. In this way, the central region may have a higher image quality and uniformity, corresponding to the regions of higher clinical interest, while doing so with a means that is robust, simple, and economical.

Moreover, it is desirable to provide a method and device that provides different rates of angular transducer movement that vary across the scanned region. For the particular case of scanning the human eye, the angular pattern of the pulsing can be symmetric about the central scan line, since the eye itself is generally symmetric about a central axis. Furthermore, embodiments described herein disclose means for providing configurable scan line densities that are suitable for implementation in mechanically scanned ultrasound imaging systems, as these predominate the ophthalmic market, for reasons noted earlier. It is further desirable that the implementation be done at a low cost, in order to serve markets and patients across the globe including those in developing nations.

Embodiments of the present disclosure are directed to systems, methods, and devices configured to detect the angular position of an ultrasound transducer and control the pulse firing pattern of the scan lines to produce a predefined sequence of scan line densities across a scan region. Embodiments include transducer devices implementing mechanical driving means that cause the transducer to move with angular motion about a pivot. Control logic is executed to determine the angular position of the transducer based on a detection means such as an encoder or the like. In response to the detected angular position of the transducer, the control logic further causes the transducer to emit an ultrasound pulse and receive a response to the emitted ultrasound pulse. That is, the control logic may be programmed to produce the pattern of scan lines (i.e., a sequence of ultrasound pulses) based on the angular position of the transducer. In particular, embodiments described enable the delivery of scan line densities that are not physically possible through mechanical means alone.

For example, as described in more detail herein, control methods and devices are configured to detect the angular position of a transducer and control the pulse firing pattern of the scan lines to produce a predefined sequence of scan line densities across a scan region. More specifically, the ultrasound transducer device and control methods are further configured to determine the angular position of the transducer based on a detection means such as an encoder and, in response to the angular position of the transducer, cause the transducer to emit an ultrasound pulse and receive a response to the emitted ultrasound pulse.

In other words, the present disclosure provides a method of implementing a scan line distribution that varies with the angular position of a mechanically scanned ultrasound system, by implementing a non-uniform angulation rate that is more constant in the central region than at the edges of the scan. The scan line distribution generated by the disclosed methods and device vary the angular position of a mechanically scanned ultrasound system by implementing a combination of a non-uniform temporal pulse rate and non-uniform angulation rate. In some embodiments, the scan line distribution may vary with the angular position of a mechanically scanned ultrasound system that is symmetric about the central scan line.

One approach to reducing the effect of high scan density at the edges of the scan where the transducer must slow down and reverse direction is to have the range of the mechanical angular motion exceed the range of the ultrasound scan. In other words, the ultrasound scan range is less than the mechanical range of the transducer angulation, so no ultrasound pulses are generated while the transducer is slowing and turning around. This has been referred to as overscanning.

Moreover, prior systems do not solve the problem of providing uniform scan line density in the central region of an ultrasound scan taken with an angulating mechanical sector ultrasound scanner at low cost and complexity because they do not allow for a transducer pulsing pattern that is specifically adapted to the requirements of an ophthalmic scan. That is, while overscanning can reduce the apparent scan line density at the edges of the scan, it requires that the mechanical system scan a larger angular range than the displayed range. This puts more demanding requirements on the mechanical system. Specifically, it must be designed for a larger angular range, and in order to maintain the same frame rate, it must angulate the transducer more quickly. Therefore, the overscanning range must be minimized.

The following will now describe scanners, systems, and methods configured to provide uniform scan line density in the central region of an ultrasound scan taken with an angulating mechanical sector ultrasound scanner, at minimal cost and complexity. For example, systems, methods, and devices described herein are configured to detect the angular position of a transducer and control the pulse firing pattern of the scan lines to produce a predefined sequence of scan line densities across a scan region by implementing an ultrasound transducer device and control logic configured to determine the angular position of the transducer based on a detection means such as an encoder. In response to determining the angular position of the transducer, a controller causes the transducer to emit an ultrasound pulse and receive a response to the emitted ultrasound pulse in a configurable pattern.

The following will now describe the systems and methods in more detail with reference to the drawings where like numbers refer to like structures.

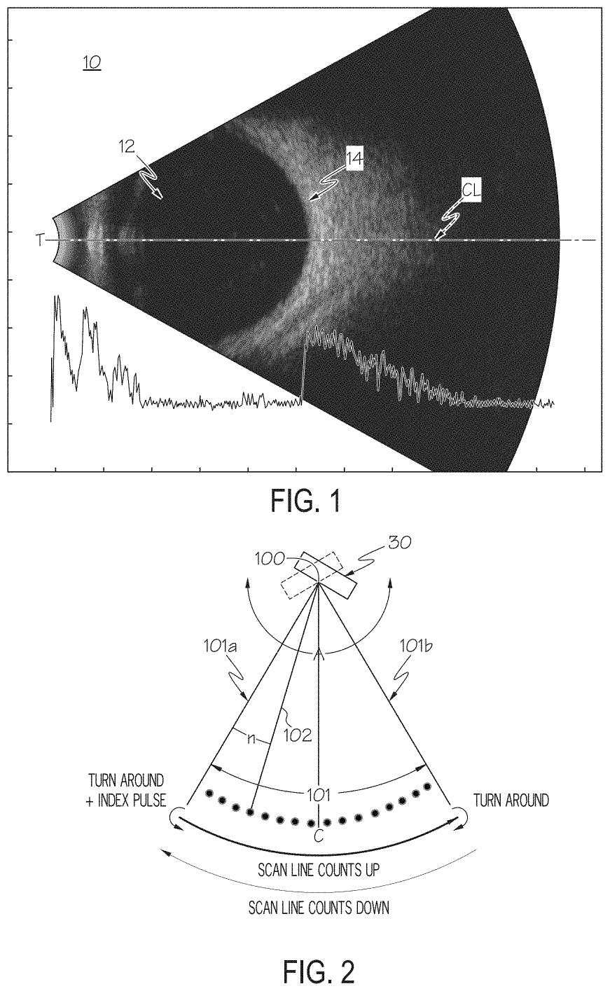

FIG. 1 is an illustrative example of an ophthalmic ultrasound image 10. The ultrasound sector scanner having a transducer is positioned (e.g., at position “T”) to the left and the eye is clearly seen to the right. The darkened circular area 12 represents the internal vitreous portion of the eye where higher resolution would be appropriate. In this particular image, the sides of the scan do not include the sclera. Note that the back of the eye 14 is circularly shaped, but the circle is not centered on the rotational pivot of the ultrasound scanner. Therefore, portions of the back of the eye at the edges of the scan are closer to the transducer than portions of the back of the eye at the center of the scan. It is also clear from this image that the image of the eye appears very symmetric about the center line “CL” of the scan, shown as a black line. Accordingly, having an ultrasound transducer device that can generate uniform scan line densities across the scan region produces a uniform ophthalmic ultrasound image. Additionally, controlling the scan line densities across the scan region may also help to increase the resolution of viewing desired aspects of the eye or other feature being scanned.

FIG. 2 shows the motional pattern of an ultrasound sector scan probe. The transducer 30 is located at position 100, the apex of the imaging sector. The transducer 30 sweeps back and forth (arrow A) over a scan region 101 as it sweeps through multiple scan angles from position 101a to 101b. As shown, it reverses its direction of angular motion or “turns around” at the edges. There are multiple ultrasound transmit/receive instances, referred to herein as an ultrasound pulse, as the transducer 30 moves from 101a to 101b. Each transmit/receive instance generates a scan line 102. The angular position (n) of each scan line 102 is required so that the ultrasound imaging system can reconstruct a two dimensional cross sectional image (as shown in FIG. 1), with the proper spatial alignment. Thus, each scan line 102 is assigned a numeric value (e.g., a scan line count (n)) that can be matched to a specific angular orientation. The scan line count may be counted upwards from the position 101a to a maximum count at position 101b. When the transducer 30 sweeps from position 101b to position 101a, the scan line count counts down from the maximum back down to zero. In this manner, scan lines that have the same angular orientation (or fall within a predefined angular orientation range) are given the same scan line count. For instance, a system which has 128 scan lines per image would start at scan line 0 at position 101a, and the scanline count would increment until the transducer 30 was oriented to position 101b, with scanline count 127. After the transducer 30 angular motion stopped and reversed (in the angular region beyond position 101b), the scanline count would resume at count 127 (when the transducer was again oriented to position 101b) and would decrement to 0 upon reaching position 101a. Implementing the systems and methods described herein, a uniform or other predefined scan line density across the scan region may be implemented. For example, the dots depicted in FIG. 2 are intended to depict a uniform distribution of scan lines across the scan region, which is achievable through the systems and methods described herein. Moreover, since generation of the scan lines is controlled by the computing device based on the determined angular position of the transducer, any number of scan line density patterns may be generated. The patterns are referred to herein as pulse firing patterns.

As the transducer 30 at position 100 is mechanically sector scanned over some total scan angle, as shown in FIG. 2, ultrasound waves are repeatedly emitted and received to produce scan lines 102 in order to create an image such as the image shown in FIG. 1.

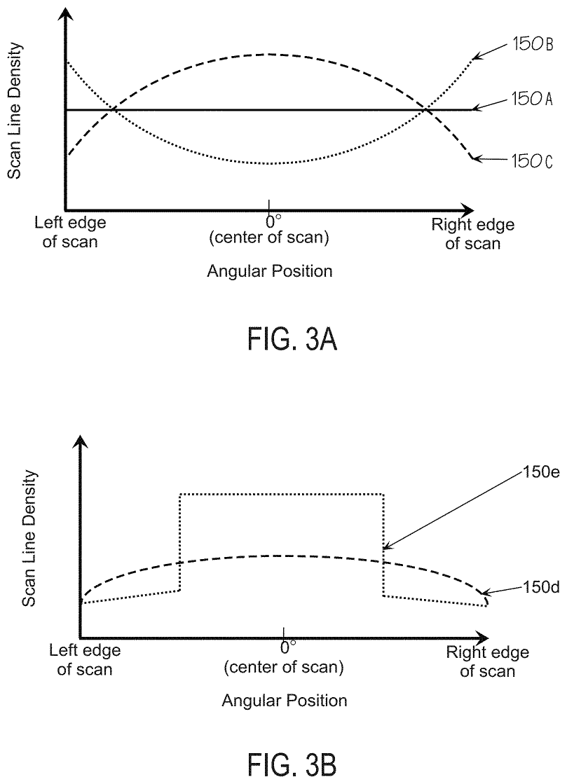

FIGS. 3A and 3B depict graphical representations of scan line densities across a scan region. For example, FIG. 3A shows a graphical representation of different scan line density plots as a function of angular position of the transducer within the scan region. The horizontal axis represents the angular position of the transducer from the left edge of the scan (corresponding to position 101a in FIG. 1) to the right edge of the scan (corresponding to position 101b in FIG. 1). There are three representations, 150a, 150b, and 150c. That is, FIG. 3A shows representations of both non-uniform representations e.g., 150b and 150c and a uniform representation 150a of scan line density curves.

Representation 150a shows a scan line distribution that is completely uniform over all angular positions of the transducer 30. Representation 150a is one that has been obtained in the past, typically obtained by varying the transducer pulsing rate as a function of scan angle to compensate for a generally undesirable mechanical scanning rate. This incurs the additional complexity of changing the pulsing rate in exact synchrony with the mechanical scanning rate, and doing so in a non-uniform manner. Clearly if the mechanical scanning rate is undesirable, i.e. it slows down at the edges of the scan in order to reverse the direction of motion and/or the pulse rate is constant with time, the result may be scan line density as depicted by representation 150b, which shows a scan line distribution that has the highest scan line density at the edges of the scan and the lowest in the center of the scan. This scan line density would be created by having a uniform transducer pulse rate and an angulation system that slows down near the edges of the scan. In some embodiments, representation 150b may be a trapezoidal, triangular, smoothed trapezoidal, smoothed triangular, sinusoidal or combination thereof. The shape most typically is of a sinusoidal nature. One specific difficulty from a sinusoidal shape is that the change in scan line density with position is never constant.

A pulse rate that completely compensates for the angular scanning rate as a function of position would produce scan line density as depicted by representation 150a. The pulse rate would in this instance be the inverse of the angular scanning rate. This would produce a scan which provides uniform scan density over the entire image. However, that is not optimal for the case of ophthalmic imaging, as noted herein, and as also noted, increases the complexity of the pulse firing, specifically because of the non-linearity of the underlying motional pattern.

Those skilled in the art would appreciate that the exact shape of the curve depicted in representations 150b and 150c are dependent on the mechanical structure and the exact firing pattern. Curves depicted in representations 150b and 150c could have a triangular shape, a trapezoidal shape, a sinusoidal shape, or some other specific shape unique to the device design. The curves depicted in representations 150b and 150c are not intended to show all possible alternatives, and those skilled in the art would appreciate that other specific shapes may be developed which do not stray from the intent of the material disclosed herein.

FIG. 3B shows another graphical representation of different scan line density plots as a function of angular position of the transducer within the scan region. The horizontal axis again represents the angular position of the transducer from the left edge of the scan (corresponding to position 101a in FIG. 1) to the right edge of the scan (corresponding to position 101b in FIG. 1). There are two representations, 150d and 150e. Representation 150d shows a scan line distribution that may be obtained by means of electronic manipulation of the pulse firing pattern. Representation 150e shows a scan line distribution that may be obtained by means of electronic manipulation of the pulse firing pattern, as it is generally not physically possible to so abruptly change the scan line density by mechanical means alone, due to the effects of inertia. Representation 150e has a uniform scan line density in the central region of the scan, which is one object of this invention. The specific pattern of representation 150e can be programmed using the methods described herein, based on an encoder output to determine the angular position of the transducer 30 as a function of rotational position of the motor, and adjusting the pulse firing pattern accordingly. Again, these representations are not all possible alternatives, and others, which may represent either mechanical means or electronic means or a combination of these two and other means are possible as well, without straying from the intent of the material disclosed herein.

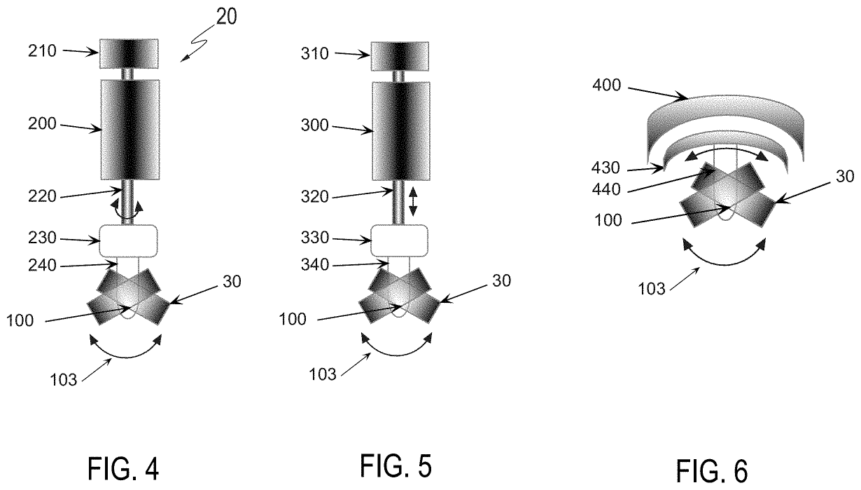

Turning now to FIGS. 4-6, example embodiments of an ultrasound device implementing different embodiments of a mechanical drive system which angulates the transducer 30 about a pivot 100, for example, driving the transducer 30 over an angular path 103 through a scan region 101.

FIG. 4 schematically depicts an ultrasound device implementing a first mechanical drive system which angulates the transducer 30 about a pivot 100, using a motor 200 which creates a rotary motion, for example, driving the transducer 30 over an angular path 103 from a rotary motor 200. Shaft 220 from motor 200 rotates and drives a mechanism 230 which converts the rotary motion into an angulation of transducer 30 via a linkage 240 shown schematically. The rotational motion of shaft 220 may be sensed using an encoder means 210. The motor 200 may be one of many types known to those skilled in the art, and may be a brushless DC motor or a stepper motor. The rotary motion of shaft 220 may be continuous in a single direction (i.e. clockwise or counterclockwise), intermittent in a single direction, or may alternate direction (e.g., clockwise, then counterclockwise, and/or repeating). The mechanism 230 may be comprised of a gear system, a pulley system, an offset pin in a disk system or any of a number of approaches as one skilled in the art would appreciate. The linkage 240 to the transducer may similarly be comprised of pulleys, gear interfaces, etc. so as to match with mechanism 230 and be able to appropriately drive transducer 30 about pivot 100.

As the rotary motor 200 rotates continuously in a single direction, the motion transfer mechanism 230 converts this motion into angular motion about the pivot 100. For example, as the motor rotates the motion transfer mechanism 230 (e.g., a cam and a cam follower) rotates causing a cam follower to angulate back and forth about a pivot in response to the interface between one or more cam follower contacts and a cam profile of the cam. Moreover, since the ultrasound transducer is coupled to the cam follower, the ultrasound transducer is configured to angulate through a scan region in response to the rotational motion provided to the motion transfer mechanism 230 (e.g., the mechanical drive system such as a cam and cam follower). In some embodiments described herein, the motion transfer mechanism 230 implements a cam and cam follower that allows the motor to operate in a continuous direction and optionally at a constant speed. This is unlike some current embodiments that use a pin on a rotating surface to effect the motional conversion, for example. In these embodiments, the angular motion of the transducer slows as it reaches the maximal angular extents depicted by positions 101a and 101b. This slowing causes the angular rate of motion to continually change over the scan region. The motion is sometimes referred to as sinusoidal. The changing angular rate of motion over the scan region, when the pulse firing pattern of the transducer is not controlled, results in undesired scan line densities across the scan region. Variability in the scan line densities across the scan region affect the level of detail that a user is able to perceive from the scan data collected by the ultrasound transducer.

Embodiments described herein are configured to cause the motion transfer mechanism 230 to operate such that the angular motion pattern may be uniform over the center of the scan region and optionally selectively controlled since through a relationship between the motor shaft position as determined by an encoder, for example, and a corresponding angular position of the ultrasound transducer. Furthermore, as described in more detail herein improvements to the motion transfer mechanism 230 such as the surface that a pin rests in, or use of a different drive linkage mechanism, such as a cam surface results in a more controllable and simpler ultrasound device. Example cam type mechanisms 230 will be described in more detail herein.

Still referring to FIG. 4, in some embodiments, the motor 200 may be a stepper motor, such that the rotary motion of shaft 220 alternately spins clockwise and counterclockwise. The speed of the motion can be controlled electronically to produce a uniform angular motion at a central portion of the scan region. However, stepper motor controlled speed and rotation can add to the complexity of the ultrasound device. In other words, this approach may require additional complex electronic control circuitry. Additionally, stepper motors may have electromagnetic noise associated and such noise is particularly troublesome with ultrasound imaging systems, as the desired received signals are very low amplitude and the spectrum of the electromagnetic noise overlaps with the spectrum of the ultrasound signal.

FIG. 5 schematically depicts another ultrasound device implementing a second mechanical drive system which angulates the transducer 30 about a pivot 100. For example, the second mechanical drive system angulates the transducer 30 about a pivot 100, using a solenoid 300 which creates an in-and-out motion. The mechanical drive system drives the transducer 30 over an angular path 103 from motion generated by the solenoid 300. Shaft 320 from solenoid 300 moves in-and-out and drives a mechanism 330 which converts said in-and-out motion into an angulation of transducer 30 via a linkage 340 shown schematically. The position and directional motion of shaft 320 may be sensed using a position detection means 310. The solenoid 300 may be one of many types known to those skilled in the art. In some embodiments, the solenoid 300 may be exchanged for a linear actuator. The motion of shaft 320 repeatedly alternates in an in-and-out direction as indicated by the bi-directional arrow. The mechanism 330 may be comprised of a gear system, a pulley system, an offset linkage system or the like. The linkage 340 to the transducer may similarly be comprised of pulleys, linkages, gear interfaces, or the like so as to match with the mechanism 330 and be able to appropriately drive transducer 30 about the pivot 100. In some embodiments, the linkages may physically connect to the transducer assembly on outer the edge(s) of the transducer rather than at the center as shown in FIG. 5. It should be understood that FIG. 5 is merely a schematic representation and particular mechanical implementations may be readily understood from the schematic representation and in view of the present disclosure.

Still referring to FIG. 5, elements including the mechanism 330 and the linkage 340, for example, convert the linear motion of shaft 320 into angular motion of transducer 30. The maximal forward extent of the shaft 320 may corresponded to scan lines 102 produced by the transducer 30 at position 101a defining an angle from center C, and the maximal rearward extent of the shaft 320 may corresponded to the scan lines 102 produced by the transducer 30 at position 101b defining an angle from center C. A sinusoidal current drive to the solenoid 300 can produce the angular motion through the scan region 101. The current drive to the solenoid 300 could also be such that the rate of angular position change is as uniform as possible over the maximum possible angular extent of transducer 30 motion through the scan region 101. In some embodiments, this approach requires additional solenoid current to apply a sufficient arresting force to reverse the momentum of the transducer and other mechanical parts. Additionally, springs may be added around the shaft 320 to take assist in arresting and changing the momentum of the solenoid motion during the reversal process.

FIG. 6 schematically depicts another ultrasound device implementing a third mechanical drive system which angulates the transducer 30 about a pivot 100. The third mechanical drive system which angulates the transducer 30 about a pivot 100, may implement a magnetic drive which magnetically couples to element 430, which through linkage 440 drives transducer 30. In some embodiments, the magnetic drive may include positional feedback elements such as induction coils configured to determine the angular position generated by the magnetic drive. Some means of position sensing may include magnetic or optical encoding means on elements 430, 440 or at position 100.

A sinusoidal drive current to the stationary element 400 produces angular motion of linkage 440 thus driving the transducer 30 back and forth through the scan region 101. The current drive to the stationary element 400 could also be such that the rate of angular position change is as uniform as possible over the possible angular extent of the transducer 30. The magnetic drive approach may require additional circuitry and drive current to apply a sufficient arresting force to reverse the momentum of the transducer and other mechanical parts as opposed to the constant rotary motion that is capable with implementation of the first mechanical drive system. Additionally, springs may be added which make contact with elements 430 or 440 at the extremes of its motion to take up the momentum and assist with the reversal process.

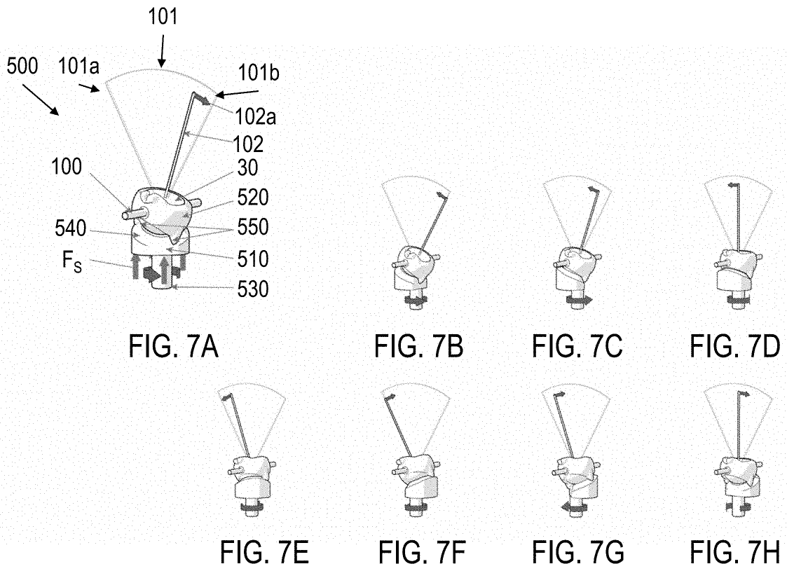

Referring now to FIGS. 7A-7H, schematic representations of a portion of an ultrasound device implementing a mechanical drive system 500 having a cam 510 and a cam follower 520 configured to receive rotational motion from a motor 200 (FIG. 4) and convert the rotational motion into angular motion that angulates the transducer 30 about a pivot 100 is depicted. Although not depicted in FIGS. 7A-7H so that the internal components may be depicted and described, a housing may enclose the mechanical drive system 500 and include a recess that receives the pivot 100. In some embodiments, the mechanical drive system 500 includes a cam 510 rotatably coupled to a shaft 530. The shaft 530 may be the shaft 220 (FIG. 4) of the motor 200 or may be a separate shaft 530 that is coupled to the shaft 220 of the motor 200. In some embodiments, the shaft 530 may be coupled to the motor 200 such that a spring force F_{S} between the motor 200 and the cam 510 may be implemented. For example, a spring or similar element may be implemented around the shaft 530 so that cam 510 may be pressed into the cam follower 520 to provide more consistent contact between the two during operation.

The cam 510 of the mechanical drive system 500 includes a cam profile 540 defining a contour on a surface of the cam 510. The cam profile 540 has a contour that corresponds to a predetermined angle that defines the scan region 101 of the transducer 30. The cam 510 further includes a socket configured to receive a ball portion of the cam follower 520. The cam 510 and the cam follower 520 may movably couple together through the socket and ball portion interface, for example, that defines a ball-and-socket type joint. The cam follower 520 may further include one or more cam follower contacts 550 extending from the ball portion or the outer surface of the cam follower 520 so that the one or more cam follower contacts 550 contact the cam profile 540 of the cam 510. As the cam 510 rotates, the one or more cam follower contacts 550 cause the rotational motion of the cam 510 to angulate the cam follower 520 back and forth about a pivot 100 based on the contour defined by the cam profile 540 of the cam 510.

In some embodiments, there may be two cam follower contacts 550 positioned opposite each other on the cam follower 520. The one or more cam follower contacts 550 may be molded or otherwise formed with the cam follower 520. While in some embodiments, the one or more cam follower contacts 550 may be a post, pin, or other extension inserted into the cam follower 520 to contact the cam profile 540. The one or more cam follower contacts 550 may be the same or a different material than the cam follower 520. However, experimentation has shown than that wear of the cam follower contacts 550 and/or the cam profile 540 may be reduced by matching the materials used to form the cam follower contacts 550 and the cam profile 540.

The cam 510 and the cam follower 520 can be made from any suitable high strength, low friction material such as High Density Polyethylene (HDPE), Teflon, or other suitable plastic. It can be molded, machined, or printed using additive manufacturing techniques. The cam 510 and the cam follower 520 may be molded, machined, or formed using an additive manufacturing process. Molding may produce a good surface finish and smooth interface between the cam 510 and the cam follower 520. Additionally, molding may be accomplished with the lowest per-part cost, at the expense of higher up front tooling costs and time. Machining may also produce a good finish but the per-part cost may be higher than molding. Additive manufacturing allows for the most rapid prototyping of new designs, but may require a post processing step to achieve a good surface finish.

The cam follower 520 further houses the transducer 30. Accordingly, angular motion of the cam follower 520 translates into angular motion of the transducer 30. As the transducer 30 angulates back and forth through a scan region 101, the transducer 30 generates scan lines 102. The pulse firing pattern of the scan lines 102 generated by the transducer 30 is controlled by a computing device 602. By way of example, when the motor 200 generates rotational motion, the shaft 530 rotates and further causes the cam 510 to rotate. An encoder 210 (FIG. 4) detects a rotational position of the shaft 530 as driven by the motor 200. The encoder 210 generates signals corresponding to the rotational position and communicates the signals to the computing device.

The computing device 602 determines the angular position of the transducer 30 within the scan region 101 based on the rotational position of the shaft 530 detected by the encoder 210. The determination may be based on a calibration record that correlates rotational positions of the shaft 530 with the angular positions of the transducer 30 coupled to the cam follower 520. In order to achieve desired scan line densities so that desired resolutions within the generated ultrasound image, it is necessary to implement control of the pulse firing pattern of the scan lines. That is, as described above, merely causing the transducer 30 to generate a scan lines at the same time interval will result in low density of scan lines in a central portion and higher densities at the edges. Additionally, attempting to control the density of scan lines by controlling the speed at which the transducer translates a scan region is complex because it requires precision motor control and circuitry to drive the same. However, as described in embodiments herein, a motor speed may be set to a constant speed and a scan line firing command may be sent by the computing device 602 to the transducer based on the determined angular position of the transducer. This eliminates the need to implement complex motor controls. In turn, present embodiments provide flexibility to operators in developing customized pulse firing patterns that can deliver uniform scanning or custom scanning procedures that may require an increase in the scan line density over a portion of the scan region to capture additional detail for particular area of interest. That is, the computing device 602 controls the pulse firing pattern of scan lines to produce a predefined sequence of scan line densities across the scan region based on the determined angular position of the ultrasound transducer.

Referring now to FIGS. 7A-7H more generally, when viewing FIGS. 7A-7H in sequence, the operation of the ultrasound device can be observed. That is starting with FIG. 7A, the arrow wrapped around the shaft 530 depicts the rotational motion of the shaft 530 and the cam 510. As FIGS. 7B-7H are subsequently observed the angular motion of the cam follower 520 about the pivot 100 can be observed. That is, in FIGS. 7A and 7B one of the cam follower contacts 550 corresponds to a low portion of the cam profile 540 while the other cam follower contact 550 correspond to the this high portion of the cam profile 540. This orientation causes the cam follower 520 to be at an angle that corresponds to the edge 101b of the scan region 101. As rotation of the cam 510 progresses in FIGS. 7C-7E, the lower portion of the cam profile 540 moves to be in contact with the opposite cam follower contact 550 as it was in FIG. 7B and similarly the higher portion of the cam profile 540 moves to be in contact with the opposite cam follower contact 550 as it was in FIG. 7B. This movement causes the cam follower 520 to sweep through the scan region 101 from one edge 101b to the other edge 101a of the scan region 101 in a direction indicated by arrow 102a. Continuing with FIGS. 7F-7H, the cam 510 continues to rotate in the same direction but the cam follower 520 causes the transducer 30 to pivot in a reverse direction such that it sweeps back through the scan region 101 from edge 101a to the other edge 101b in a direction indicated by arrow 102a. The oscillation of the cam follower 520 may continue as the cam 510 continues to rotate. Furthermore, as the cam follower 520 and the transducer 30 oscillate, the computing device 602 is controlling when to generate a scan line 102 based on the angular position of the cam follower 520.

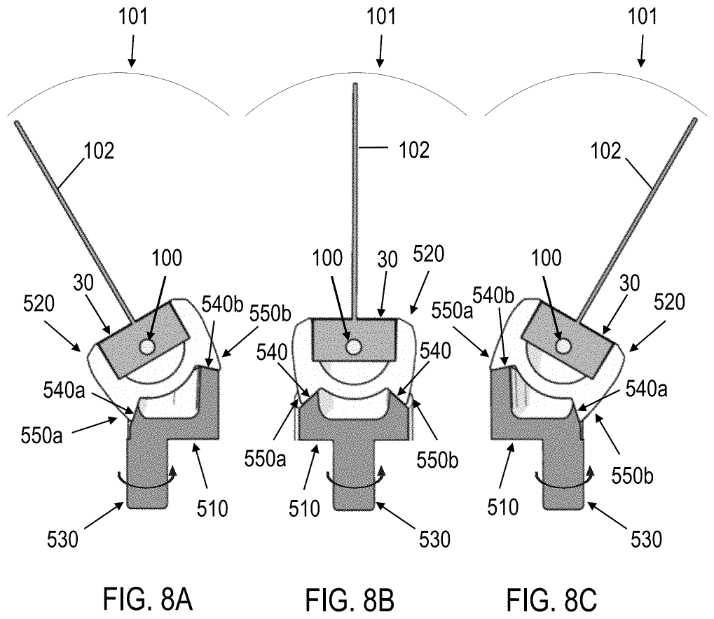

Referring now to FIGS. 8A-8C, cross-sectional view of the portion of the ultrasound device depicted in FIGS. 7A-7H is depicted. In particular, FIGS. 8A-8C depict interactions between the cam 510 and the cam follower 520 as the shaft 530 rotates. As discussed with reference to FIGS. 7A-7H, the cam 510 includes a cam profile 540 that defines a contour that when placed in contact with the cam follower contacts 550a and 550b of the cam follower 520 cause the cam follower 520 angulate back and forth about the pivot 100. The pivot 100 is rotatably supported within a housing. That is, the pivot 100 prevents the cam follower 520 from rotating with the cam 510 and instead facilitates the conversion of rotational motion of the cam 510 to angular motion of the cam follower 520. Referring to FIG. 8A, the cam profile 540 includes a first portion 540a that is offset from a second portion 540b. The first and second portions 540a and 540b are portions of the overall contour that defines the cam profile 540. The cam follower 520, as discussed briefly with respect to FIG. 7A, includes one or more cam follower contacts 550. The cam follower 520 depicted in FIG. 8A includes two cam follower contacts 550a and 550b. The cam follower contacts 550a and 550b may be formed as contiguous members of the cam follower 520. In some embodiments, the cam follower contacts 550a and 550b may be secondary elements attached to the cam follower 520, such as a pin or rod comprising a material that is different than the cam follower 520. The secondary element configuration may allow the cam follower contacts 550a and 550b to be adjusted for custom interfacing with the cam profile 540.

As depicted in FIG. 8A, the cam 510 is positioned, for example, at a 0-degree orientation where the first portion 540a of the cam profile 540 of the cam 510 is oriented to the left and the second portion 540b of the cam profile 540 of the cam 510 is oriented to the right in the depicted cross-section. Additionally, the first cam follower contact 550a is in contact with the first portion 540a and the second cam follower contact 550b is in contact with the second portion 540b. When the cam 510 is position in the 0-degree orientation the cam follower 520 is angled to the left (i.e., with reference to the perspective show in FIG. 8A). As the shaft 530 rotates, for example, from the 0-degree orientation depicted in FIG. 8A to a 90-degree orientation depicted in FIG. 8B, the first and second portions 540a and 540b of the cam profile 540 rotate causing the cam follower 520 to angulate from a left position as depicted in FIG. 8A to the right and into a central scan region 101 position as depicted in FIG. 8B. Furthermore, as the shaft 530 continues to rotate, the cam 510 continues to rotate from the 90-degree orientation depicted in FIG. 8B to a 180-degree orientation depicted in FIG. 8C. Moreover, the first and second portions 540a and 540b of the cam profile 540 rotate causing the cam follower 520 to continue to angulate the central scan region 101 position as depicted in FIG. 8B to a right position of the scan region 101 as depicted in FIG. 8C. When the cam 510 is rotated to the 180-degree orientation depicted in FIG. 8C, the first portion 540a of the cam profile 540 is positioned in contact with the second cam follower contact 550b and the second portion 540b of cam profile 540 is positioned in contact with the first cam follower contact 550a. As the shaft 530 continues to rotate, the cam 510 rotates with the shaft 530 and causes the cam follower 520 to reverse its motion from the right position of the scan region 101 back to the left position of the scan region 101.

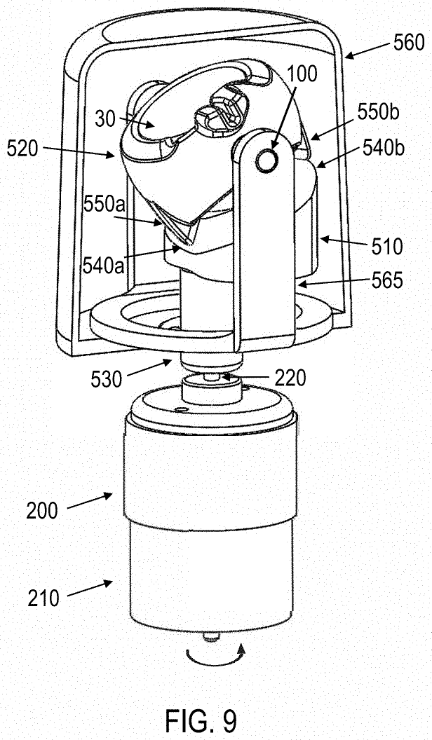

Turning to FIG. 9, an illustrative schematic of a partial assembled view of the ultrasound device is depicted. FIG. 9, with reference to FIGS. 7A-7H and 8A-8C, now depicts an example housing structure 560 that is configured to enclose the ultrasound transducer 30 and the mechanical drive mechanism depicted and described with reference to FIGS. 7A-7H and 8A-8C. The housing structure 560 includes an outer housing that may be configured to contact the eye of a patient or other feature for scanning. The housing further includes an internal structure 565 having one or more mounting arms that rotatably couple the cam follower 520 via the pivot 100 to the internal structure 565 of the housing 560. The internal structure 565 and housing 560 prevent the cam follower 520 from rotating with the cam 510 and shaft 530. The housing 560 may be supported around the shaft 530 by a set of bearings that allow the shaft 530 to rotate independently from the housing 560. Further depicted in FIG. 9, the shaft 530 is coupled to the motor shaft 220 of the motor 200. The motor 200 may be further coupled to an encoder 210 which monitors and relays the rotational position of the motor shaft 220 to the controller. In some embodiments, although not depicted in FIG. 9, a further housing is provided around the motor such that a user may hold and manipulate the position of the ultrasound device.

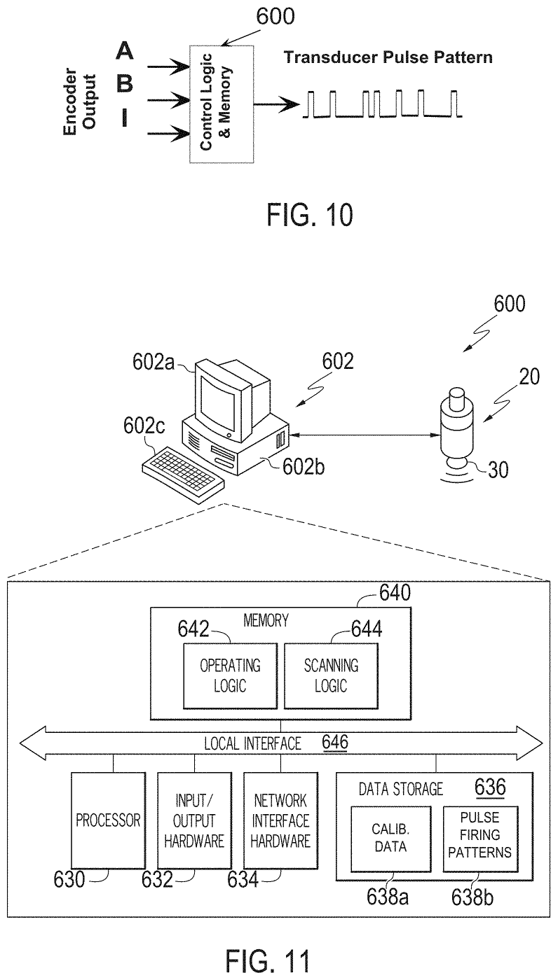

Referring now to FIG. 10, a block diagram block diagram of a means of using encoder signals to produce a transducer pulse firing pattern that is corresponds to the angular position of the transducer is depicted. By incorporating the index signal I from the encoder output, with the quadrature signals A and B, the control logic of the computing device 602 can determine the angular position of the transducer 30. That is, since the rotational position of the motor 200 is detected by the encoder signals, the control logic and memory can be configured to produce control signals that cause the transducer to implement a predefined pulse firing pattern of scan lines with respect to angular position. The pattern can be uniform in time, with a constant Pulse Repetition Frequency, or can correspond to uniform angular increments of the transducer, or can correspond to arbitrary angular increments of the transducer, which may be specified by a user through a control interface.

Referring now to FIG. 11, an illustrative ultrasound system 600 is depicted. The ultrasound system includes an ultrasound scanning probe having transducer 30 communicatively coupled to a computing device 602. The transducer 30 is any device capable of emitting and receiving ultrasound signals. The transducer 30 may be configured to mechanically oscillate over a predefined angular region to enable an ultrasound scan of material such as an eye. The transducer 30 may also be configured to sweep through the predefined angular region through electronic means independent of or in conjunction with mechanical means such as a pivot apparatus within the ultrasound scanning probe or the like. Whether the transducer 30 is mechanically and/or electronically driven to scan an angular region, the transducer 30 is configured to emit and/or receive an ultrasound signal at a predefined frequency and position, which may be controlled and/or tracked by the computing device 602. In other words, the angular position of the transducer 30 is determined and tracked by the computing device 602. Moreover, as described above, the angular position of the transducer 30 is utilized to implement a predefined pulse firing pattern to generate a desired scan lines densities across the scan region.

The computing device 602 may be used to control the transducer and/or receive signals from the transducer to generate ultrasound images. The computing device 602 may be a convention computer or any other electronic control unit capable of controlling the transducer 30 to produce ultrasound images according to the embodiments disclosed herein. As depicted and described herein, the computing device 602 may utilize hardware, software, and/or firmware, according to embodiments shown and described herein. While in some embodiments, the computing device 602 may be configured as a general-purpose computer with the requisite hardware, software, and/or firmware, in some embodiments, the computing device 602 may be configured as a special purpose computer designed specifically for performing the functionality described herein.

The computing device 602 may include a display 602a, a processing unit 602b and an input device 602c. The display 602a may be a touchscreen interface or any other display capable of presenting data and/or images to a user. The input device 602c may be a keyboard, mouse, stylus, touchpad or the any other hardware device capable of translating user action into a computing command. The computing device 602 may include a processor 630, input/output hardware 632, network interface hardware 634, a data storage component 636, which store calibration data 638a, pulse firing patterns 638b, and other ultrasound data, and a memory component 640. The memory component 640 may be machine-readable memory (which may also be referred to as a non-transitory processor readable memory). The memory component 640 may be configured as volatile and/or nonvolatile memory and, as such, may include random access memory (including SRAM, DRAM, and/or other types of random access memory), flash memory, registers, compact discs (CD), digital versatile discs (DVD), and/or other types of storage components. Additionally, the memory component 640 may be configured to store operating logic 642, scanning logic 644 (each of which may be embodied as a computer program, firmware, or hardware, as an example). A local interface 646 is also included in FIG. 11 and may be implemented as a bus or other interface to facilitate communication among the components of the computing device 602.

The processor 630 may include any processing component(s) configured to receive and execute programming instructions (such as from the data storage component 636 and/or the memory component 640). The instructions may be in the form of a machine-readable instruction set stored in the data storage component 636 and/or the memory component 640. The processor 630 may also referred to herein as an electronic control unit. The input/output hardware 632 may include a monitor, keyboard, mouse, printer, camera, microphone, speaker, and/or other device for receiving, sending, and/or presenting data. The network interface hardware 634 may include any wired or wireless networking hardware, such as a modem, LAN port, Wi-Fi card, WiMax card, mobile communications hardware, and/or other hardware for communicating with other networks and/or devices.

It should be understood that the data storage component 636 may reside local to and/or remote from the computing device 602 and may be configured to store one or more pieces of data for access by the computing device 602 and/or other components. As illustrated in FIG. 11, the data storage component 636 stores calibration data 638a and pulse firing patterns 638b. The calibration data 638a may include correlations between rotational positions of the motor 200 and the angular positions of cam follower 520 (i.e., the transducer 30). Calibration may be required since the cam profile 540 may vary slightly between ultrasound devices. The pulse firing patterns 638b are scan line density patterns that are desired for a particular scanning operation. Some examples were presented with respect to FIGS. 3A and 3B.

Still referring to FIG. 11, included in the memory component 640 are the operating logic 642 and scanning logic 644. The operating logic 642 may include an operating system and/or other software for managing components of the computing device 602. The scanning logic 644 may be logic configured to carry out an ultrasound scan and control the generation of scan lines by a transducer based on a pulse firing pattern and determined angular position of the transducer. The scanning logic 644 may also be configured to enable a user to define or customize pulse firing patterns or other properties and/or functions of the ultrasound system 600.

It should be understood that the components illustrated in FIG. 11 are merely exemplary and are not intended to limit the scope of this disclosure. More specifically, while the components in FIG. 11 are illustrated as residing within the computing device 602, this is merely an example. In some embodiments, one or more of the components may reside external to the computing device 602.

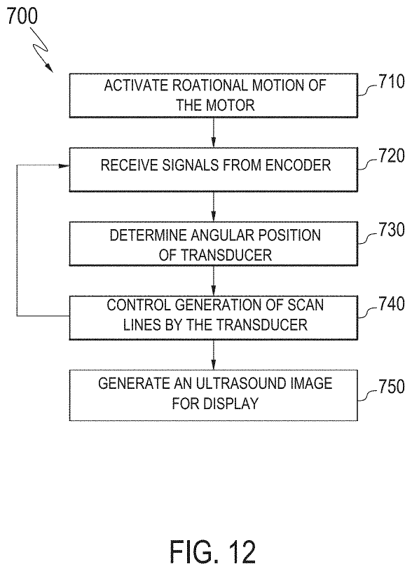

Referring now to FIG. 12, an illustrative method of implementing an ultrasound scan using the systems and devices described herein is depicted. As described above, the method may be carried out by a computing device 602. The flow diagram 700 depicted in FIG. 12 is a representation of a machine-readable instruction set stored in the non-transitory computer readable memory 640 (FIG. 11) and executed by the processor 630 (FIG. 11) of the computing device 602. The process of the flow diagram 700 in FIG. 12 may be executed at various times and repeated with various types of environments.

At block 710, the computing device 602 may transmit a signal to the motor 200 to active and operate a predetermined speed. The rotational motion of the motor 200 causes the cam 510 to rotate and thereby the cam follower 520 to angulate back and forth through a scan region. At block 720, the computing device 602 receives one or more signals from the encoder. The encoder signals corresponded to positions that the encoder detects. At block 730, the encoder signals are analyzed by the computing device 602 to determine the angular position of the ultrasound transducer within the scan region. In some embodiments the encoder signals may correspond to the rotational position of the motor, while in others the encoder signals may correspond to an angle measurement of the cam follower 520 for example based on a position of the pivot 100. In embodiments, where the encoder signals correspond to the rotational positon of the motor, the computing device 602 may utilize a calibration data that correlates the rotational position of the motor 200 to the angular position of the transducer 30. Once the angular position of the transducer 30 is determined, at block 740, the computing device 602 implements control of when the transducer 30 should generate a scan line. For example, the computing device 602 generates and sends a control signal to the transducer 30 causing the transducer 30 to generate a scan line when the computing device 602 determines that the transducer 30 is at a predefined angular position as defined, for example, by the pulse firing pattern. The computing device 602 continues to control generation of scan lines from the transducer 30 based on the angular position in a loop. As the transducer generates scan lines and receives responses from the generated scan lines, the computing device 602, at block 750 may generate an ultrasound image for display.

The functional blocks and/or flowchart elements described herein may be translated onto machine-readable instructions or as a computer program product, which when executed by a computing device, causes the computing device to carry out the functions of the blocks. As non-limiting examples, the machine-readable instructions may be written using any programming protocol, such as: descriptive text to be parsed (e.g., such as hypertext markup language, extensible markup language, etc.), (ii) assembly language, (iii) object code generated from source code by a compiler, (iv) source code written using syntax from any suitable programming language for execution by an interpreter, (v) source code for compilation and execution by a just-in-time compiler, etc. Alternatively, the machine-readable instructions may be written in a hardware description language (HDL), such as logic implemented via either a field programmable gate array (FPGA) configuration or an application-specific integrated circuit (ASIC), or their equivalents. Accordingly, the functionality described herein may be implemented in any conventional computer programming language, as pre-programmed hardware elements, or as a combination of hardware and software components.

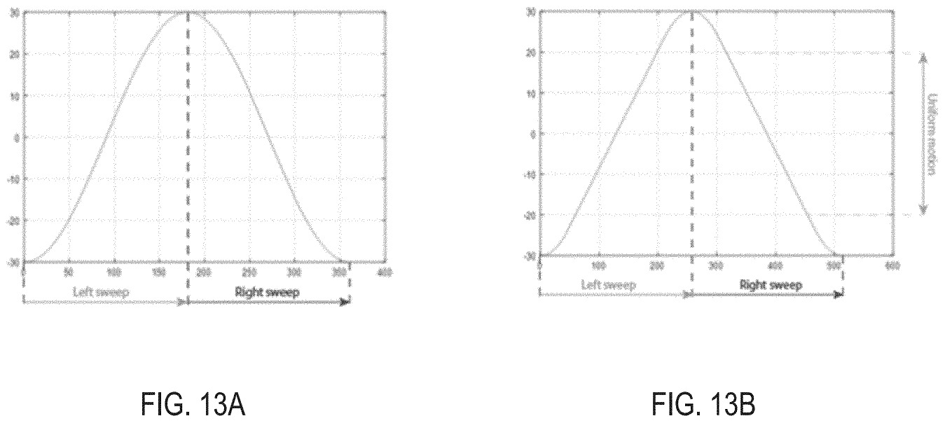

Referring now to FIGS. 13A-13B, illustrative plots depicting the distribution of pulse firing events using a typical mechanical system and the electro-mechanically controlled ultrasound scan density device according to embodiments depicted and described herein. In particular, FIG. 13A depicts a plot of pulse firing events using the typical mechanical system. The pulse firing events from left (−30 degrees) to right (+30 degrees) and then right (+30 degrees) to left (−30 degrees) has an uneven (e.g., a sinusoidal) distribution especially through the central portion, for example, between −20 degrees and +20 degrees. The graph shows the relationship between the angle of the motor shaft 220 and the angular position of the transducer 30. At no point in the left graph is there a linear relationship between the two angles.

Referring to FIG. 13B, a plot depicting the electro-mechanically controlled ultrasound scan density device disclosed herein where the central region (e.g., −20 degrees to +20 degrees) has uniform angular motion with respect to time, and a linear relationship between the angle of the transducer 30 and the motor shaft 220. The scan parameters can further be configured such that the region of uniform motion corresponds to the desired visual scan angle of the system and the areas of “turnaround” can be in the overscan region.

Within the central region of uniform motion, a constant pulse rate can be produced having uniformly spaced scan lines with constant angular offset between scan lines. If it is desired to have further manipulation of the scan line density, it is easily accomplished because the design has already provided for uniform angular motion in this central region. This simplifies the selection of higher scan line densities, as would be appreciated by one skilled in the art. There is no need to first determine the temporal compensation required to overcome the sinusoidal, or other non-uniform scanning motion, before applying the desired scan line density pattern.

It should now be understood that embodiments of the present disclosure include ultrasound systems, ultrasound devices and methods of operating the same. In some embodiments, the ultrasound system includes a computing device and an ultrasound transducer configured to angulate through a scan region in response to a mechanical drive system that converts rotational motion generated by a motor into angular motion that angulates the ultrasound transducer through the scan region, and an encoder configured to detect a rotational position of a shaft of the motor. The computing device is configured to determine an angular position of the ultrasound transducer within the scan region based on the rotational position of the shaft detected by the encoder, and control generation of scan lines from the transducer based on a pulse firing pattern of scan lines to produce a predefined sequence of scan line densities across the scan region and the determined angular position of the ultrasound transducer.

In one embodiment the A, B, and I signals are used to sequence through digital memory locations, said memory locations containing a transducer pulse pattern. Said transducer pulse pattern may be created to represent the desired correspondence between angular positions and scan lines, over the entire range on the angular scan from position 101a to 101b.

In another embodiment, a microprocessor is programmed to evaluate the position signals from the encoder, and using an algorithm, to produce the appropriate transducer pulse pattern. The embodiments which include either digital memory locations or a microprocessor (or a combination of both) also afford the capability to change the scan line density configuration during the operation of the system. In contrast, embodiments which use mechanical drive modifications are generally fixed at the time of design and cannot be changed during operation.

The embodiments which include either digital memory locations or a microprocessor (or a combination of both) also afford the capability to selectively increase the scan line density, and therefore the image quality, of a region that is not centered on the image, but is selected by the user. This would be useful in the case of an area of interest, for example, a tumor or foreign object imbedded it the eye, which is offset from the center line. The user of the instrument or system could select via a software user interface that region which should have higher resolution, and the system could increase the scan line density in that region.

In any embodiment, the image reconstruction algorithm must adapt to the exact angular scan line positions so that there is no distortion of the reconstructed image.

Further, in any embodiment, the designer must account for the potentially finite data transfer rates from the transducer and analog signal electronics through digitization and transfer to the imaging system. This may limit the total number of scan lines permitted within a scanning sequence, and therefore higher scan line densities must be chosen judiciously.

The results of modifications to the mechanical drive mechanisms or to the transducer pulse patterns, as described, can produce nearly any desired scan line density configuration, as shown in FIG. 3A, representation 150a, or FIG. 3B, representations 150d or 150e. It is clear to one skilled in the art, that the desired scan line density configuration may be different for different specific implementations, for instance as a function of the ultrasound beam parameters, the actual dimensional characteristics of the eye being examined, etc. Those skilled in the art would also realize that there are limits to the implementation of different scan line density configurations based on mechanical limitations, for instance the rapidity with which the transducer can reverse direction. Those skilled in the art would further realize that the optimal scan line density configuration may best be obtained by a combination of both mechanical means (mechanical drive mechanism) and electronic means (transducer pulse patterns).

It should be further understood that, the described embodiments provide for increased scan line density, and thereby higher image quality, in a region of the ultrasound scan, hereby affording improved clinical utility. The scan line density is increased in the specified region, at the expense of other regions where there is less clinical interest. This assumes that there are a finite number of scan lines available for transmission, reception, processing and display, as is common with any electronic system. Specifically, there are data transfer limitations, especially with more cost sensitive system, and moreover this is the case with systems based on, for example, USB connection schemes. For example, assuming a frame rate (i.e., the rate at which complete scans are displayed to the user) of 12-24 frames per second, and a typical total number of scan lines of 256, there are limits to the total data transfer rate. Therefore, it may not be feasible to simply increase the total number of scan lines to say, 512, in order to provide the additional image quality required for a limited portion of the scan. In such a case, it may be better to change the scan line density selectively across the image while maintaining the same overall number of scan lines.

Furthermore, structurally, the solution proposed herein has either modified mechanical scan means, which permit uniform angulation over a portion of the scan, or a combination of the modified mechanical scan means with a modified electronic means, which permit faster transducer firing over a portion of the scan. Mechanical means, as noted, can be a change to the motor drive or a change to the mechanical interface between a motor and the angulation mechanism. Electronic means can mean a change to the transducer pulse pattern based on a fixed pattern relative to the angular position of the transducer, or a changeable pattern using a microcontroller, a memory circuit, or a combination of similar means. Further, the implementation may comprise a combination of mechanical and electronic means, especially if the mechanical means provide a baseline of improved transducer motion, and the electronic means then becomes an additional adjustment available to the operator of the system.

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present disclosure, including, but not limited to, embodiments defined in the appended claims. More specifically, although some aspects of the present disclosure are identified herein as preferred or particularly advantageous, it is contemplated that the present disclosure is not necessarily limited to these aspects.

What is claimed is:

1. An ultrasound system comprising a computing device, a transducer configured to angulate through a scan region in response to a mechanical drive system that converts rotational motion generated by a motor into angular motion that angulates the transducer through the scan region, and an encoder configured to detect a rotational position of a shaft of the motor, wherein the computing device is configured to:
determine an angular position of the transducer within the scan region based on the rotational position of the shaft detected by the encoder, and
control generation of scan lines from the transducer based on a pulse firing pattern of scan lines to produce a predefined sequence of scan line densities across the scan region and the determined angular position of the transducer.

2. The ultrasound system of claim 1, wherein the predefined sequence of scan line densities across the scan region includes a greater scan line density in a central portion of the scan region than a region corresponding to at least one edge of the scan region.

3. The ultrasound system of claim 1, wherein the scan lines generated by the transducer at or about edges of the scan region are generated at longer intervals than the scan lines generated in a central portion of the scan region without increasing or decreasing a speed of the rotational motion of the motor.

4. The ultrasound system of claim 1, wherein one rotation of the shaft of the motor causes the transducer to angulate back and forth once through the scan region.

5. The ultrasound system of claim 1, wherein the predefined sequence of scan line densities across the scan region is uniform within a predefined tolerance.

6. The ultrasound system of claim 5, wherein a speed of the rotational motion of the motor is maintained at a predefined speed.

7. The ultrasound system of claim 1, wherein the mechanical drive system comprises:
a cam rotatably coupled to the shaft of the motor, the cam comprising a cam profile defining a contour about a socket, the socket configured to receive a ball portion of a cam follower,
one or more cam follower contacts positioned on the cam follower to contact the cam profile of the cam, wherein as the shaft of the motor rotates, the cam rotates causing the cam follower to angulate back and forth about a pivot in response to an interface between the one or more cam follower contacts and the cam profile of the cam.

8. The ultrasound system of claim 7, wherein the transducer is coupled to the cam follower.

9. A method of controlling an ultrasound device comprising a transducer configured to angulate through a scan region in response to a mechanical drive system that converts rotational motion generated by a motor into angular motion that angulates the transducer through the scan region, and an encoder configured to detect a rotational position of a shaft of the motor, the method comprising:
determining an angular position of the transducer within the scan region based on the rotational position of the shaft detected by the encoder; and
controlling generation of scan lines from the transducer based on a pulse firing pattern of scan lines to produce a predefined sequence of scan line densities across the scan region and the determined angular position of the transducer.

10. The method of claim 9, wherein the predefined sequence of scan line densities across the scan region includes a greater scan line density in a central portion of the scan region than a region corresponding to at least one edge of the scan region.

11. The method of claim 9, wherein the scan lines generated by the transducer at or about edges of the scan region are generated at longer intervals than the scan lines generated in a central portion of the scan region without increasing or decreasing a speed of the rotational motion of the motor.

12. The method of claim 9, wherein the predefined sequence of scan line densities across the scan region is uniform.

13. The method of claim 9, wherein a speed of the rotational motion of the motor is maintained at a predefined speed and a distribution of scan lines across an arc of the scan region is uniform within a predefined tolerance.

14. The method of claim 9, wherein one rotation of the shaft of the motor causes the transducer to angulate back and forth once through the scan region.

15. The method of claim 9, wherein the mechanical drive system comprises:
a cam rotatably coupled to the shaft of the motor, the cam comprising a cam profile defining a contour about a socket, the socket configured to receive a ball portion of a cam follower,
one or more cam follower contacts positioned on the cam follower to contact the cam profile of the cam, wherein as the shaft of the motor rotates, the cam rotates causing the cam follower to angulate back and forth about a pivot in response to an interface between the one or more cam follower contacts and the cam profile of the cam and the transducer is coupled to the cam follower.

16. An ultrasound device comprising:
a motor having a shaft configured to generate rotational motion;
an encoder configured to detect a rotational position of the shaft of the motor;
a cam rotatably coupled to the shaft of the motor, the cam comprising a cam profile defining a contour about a socket, the socket configured to receive a ball portion of a cam follower;
one or more cam follower contacts positioned on the cam follower to contact the cam profile of the cam; and
a transducer coupled to the cam follower, the transducer configured to angulate through a scan region in response the rotational motion generated by the motor that causes the cam to rotate and the cam follower to angulate back and forth about a pivot in response to an interface between the one or more cam follower contacts and the cam profile of the cam.

17. The ultrasound device of claim 16, further comprising a controller communicatively coupled to the encoder and the transducer, wherein the controller is configured to:
determine an angular position of the transducer within the scan region based on the rotational position of the shaft detected by the encoder; and
control generation of scan lines from the transducer based on a pulse firing pattern of scan lines to produce a predefined sequence of scan line densities across the scan region and the determined angular position of the transducer.

18. The ultrasound device of claim 17, wherein one rotation of the shaft of the motor causes the transducer to angulate back and forth once through the scan region.

19. The ultrasound device of claim 17, wherein a speed of the rotational motion of the motor is maintained at a predefined speed and a distribution of scan lines across an arc of the scan region is uniform within a predefined tolerance.

20. The ultrasound device of claim 17, wherein the predefined sequence of scan line densities across the scan region includes a greater scan line density in a central portion of the scan region than a region corresponding to at least one edge of the scan region.
